(19) United States
(12) Plant Patent Application Publication
(10) Pub. No.: US 2007/0016995 P1
(43) Pub. Date: Jan. 18, 2007

(54) Kalanchoe plant named 'Forever Maxi Light Orange'

(75) Inventor: Lyndon W. Drewlow, Lompoc, CA (US)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/177,057

(22) Filed: Jul. 8, 2005

(51) Int. Cl. A01H 5/00

(52) U.S. Cl. PLT/335

(57) ABSTRACT

‘Forever Maxi Light Orange’ is a new variety of kalanchoe that exhibits numerous and large pumpkin orange colored flowers.

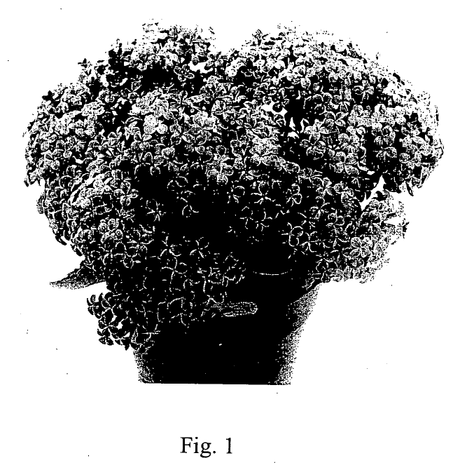

### BOTANICAL CLASSIFICATION

Kalanchoe blossfeldiana

### VARIETAL DENOMINATION

‘Forever Maxi Light Orange’

### BACKGROUND OF THE INVENTION

The present invention comprises a new and distinct cultivar of Kalanchoe blossfeldiana known by the varietal name ‘Forever Maxi Light Orange’. The new variety was discovered in Lompoc, Calif. The new variety is a result of cross breeding Seedling #98-285-1 (female parent) (unpatented) and Seedling #98-311-4 (male parent) (unpatented). The purpose of the breeding program was to develop a highly self-branching cultivar with pumpkin orange colored flowers and an early response time for the 15 cm. potted plant market. Comparisons between the new variety and its parents cannot be made, as the parental varieties no longer exist. The new variety was first asexually reproduced by tip cuttings in Lompoc, Calif. The new variety has been trial and field tested and has been found to retain its distinctive characteristics and remain true to type through successive propagations. Characteristics that distinguish the new variety from others known to the breeder include:
Semi-tall growth combined with good lateral and basal branching allows cultivar to be finished in a 12.5 cm. pot from one cutting;
Does not heat delay when night temperatures are above 22° C. during short day treatment to induce flowering;
Excellent flower coverage on upper half of plant due to excellent lateral and basal branching;
No or minimum use of growth regulators needed to grow this cultivar;
Long keeping quality of cultivar in part due to the fact the inflorescence is a cyme with an indeterminant growth habit;
Pumpkin orange flowers making it an excellent cultivar to grow for the fall season;
Tolerance to common insect and disease problems;
Large flowers for a kalanchoe at 18 mm in diameter;
Highly self-branched and basal branching without a pinch; and
Resistant to streaking of flower petals under certain conditions (No one has determined what these environmental conditions are). If streaking occurs, the cultivar is unsaleable.

The new variety was grown in a 15 cm. container in Lompoc, Calif. in a greenhouse under Dynaglass with 30% shade. The new variety initiates roots in 10 days in the summer and 12 days in the winter at 21° C. The new variety exhibits developed roots at 21 days at an average temperature of 21° C. Its rooting habit is dense and freely branching with fine and fibrous roots.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Lompoc, Calif. The color readings were taken indoors under 3500-4000 foot candles of light. The new variety was twelve weeks from the start of short days when described. Color references are primarily to the 1966 R.H.S. Colour Chart of The Royal Horticultural Society of London.

PLANT

| | |
|---|---|
| Form: | Upright with top half with flowers. |
| Height (from soil to top of flowers): | 18-20 cm. |
| Plant diameter: | 28-30 cm. |
| Vigor: | Very vigorous. |
| Branching habit: | Highly self-branched. |
| Main stem: | |
| Color: | 146C. |
| Texture: | Glabrous. |
| Length: | 10-12 cm. |
| Diameter: | 12-14 mm near soil line. |
| Internode length: | 2-3 cm and varies with |
| | location on stem. |
| Strength: | Strong |
| Lateral branches: | |
| Color: | 146C. |
| Texture: | Glabrous. |
| Length: | |
| Basal: | 12-16 cm. |
| Upper 10-13 cm. | |
| Diameter: | 4-5 mm |
| Internode length: | 10-15 cm., but highly effected |
| | by the environment. |
| Strength: | Strong. |
| Quantity: | 8-10. |
| Foliage: | |
| Arrangement: | Opposite. |
| Number of leaves per stem: | |
| Main stem: | 10-12. |
| Laterals: | 2-4. |
| Size of leaf: | |
| Length: | 9-10 cm. |
| Width: | 5-6 cm. |
| Shape of leaf (generally): | Ovate. |
| Shape of apex: | Obtuse. |
| Shape of base: | Acute to rounded. |
| Texture: | Glabrous. |
| Aspect: | 90° to main stem, hanging |
| | down slightly. |
| Margin type: | Crenate and shallow lobed. |
| Fragrance: | None. |
| Color: | |
| Young leaves: | |
| Upper surface: | 146B. |
| Lower surface: | 146C. |
| Mature leaves: | |
| Upper surface: | 147A. |
| Lower surface: | 147B. |
| Petiole: | |
| Length: | 15 mm and can vary. |
| Diameter: | 6 mm. |
| Color: | 146B. |
| Texture: | Glabrous. |
| Veins: | |
| Venation pattern: | Pinnate. |
| Color: | |
| Upper surface: | 147A. |
| Lower surface: | 147B. |

FLOWER

| | |
|---|---|
| Natural flowering season at | Early January in Lompoc, California. |
| specified location: | |
| Flower type and habit: | Epigenous, terminal flower on main |
| | stem opens first, followed by |
| | terminal buds on laterals. |
| Number of flowers per plant: | 250 or more. |
| Time of day of flower opening: | Morning. |
| Fragrance: | None. |
| Longevity: | Up to 2 weeks, but the environment |
| | the plant is in will affect it. |
| Flower height: | 15 mm, including corolla tube. |
| Flower diameter: | 18 mm. |
| Bud: | |
| Stage of development when | Mature, just before opening. |
| described: | |
| Shape: | Oblong. |
| Diameter: | 4 mm. |
| Length: | 13 mm. |
| Color: | 146C at the calyx area and |
| | 31B at the tip. |
| Corolla: | |
| Diameter: | 18 mm. |
| Depth: | 14 mm. |
| Tube: | |
| Length: | 7 mm. |
| Diameter: | 4 mm. |
| Petals: | |
| Arrangement: | Whorl of 4 fused. |
| Quantity: | 4. |
| Length: | 9 mm at the non-fused part. |
| Width: | 8 mm. |
| Shape: | Ovate to rounded. |
| Apex: | Cuspidate. |
| Base: | Rounded. |
| Margin: | Entire. |
| Texture: | Smooth, soft. |
| Color: | |
| Upper surface: | 30C. |
| Lower surface: | 35B. |
| Sepals: | |
| Quantity: | 4. |
| Shape: | Lanceolate. |
| Length: | 6 mm. |
| Width: | 2 mm. |
| Color: | 146D. |
| Peduncles (main stem): | |
| Length: | 16 mm. |
| Diameter: | 4 mm. |
| Color: | 146C. |
| Texture: | Glabrous. |
| Orientation: | 45° Angle. |
| Strength: | Strong. |
| Pedicels: | |
| Length: | 6 mm that can vary. |
| Width: | 2 mm. |
| Color: | 146C. |
| Disease resistance: | Resistant. |
| Weather tolerance: | Tolerant. |
| Lasting quality: | |
| On plant: | 4-6 weeks if receiving proper care. |
| Of cut flowers: | Not usually used as a cut flower. |
| Seed production and description: | Not applicable. |
| Fruit production and description: | Not applicable. |

REPRODUCTIVE ORGANS

| | |
|---|---|
| Stamens: | |
| Number (per flower): | 8. |
| Filament length: | Less than 1 mm. |
| Anthers: | |
| Shape: | Flat; elliptical. |
| Length: | Less than 1 mm. |
| Color: | Yellow green. |
| Pollen: | |
| Color: | Yellow. |
| Amount: | Abundant. |
| Pistils: | |
| Length: | 10 mm. |
| Styles: | |
| Length: | 4 mm. |
| Color: | Light yellow-green. |
| Stigma: | |
| Shape: | Flat. |
| Color: | White when mature. |
| Ovary color: | 146B. |

I claim:

1. A new and distinct variety of kalanchoe named ‘Forever Maxi Light Orange’ as described and illustrated.
